(19) United States
(12) Patent Application Publication
(10) Pub. No.: US 2009/0222086 A1
(43) Pub. Date: Sep. 3, 2009

(54) Resorbable Cornea Button

(76) Inventors: Ge Ming Lui, San Francisco, CA (US); Anthony Lee, San Francisco, CA (US); Shaossheng Dong, Honolulu, HI (US); Hank Wuh, Honolulu, HI (US)

Correspondence Address: JACOBSON HOLMAN PLLC, 400 SEVENTH STREET N.W., SUITE 600, WASHINGTON, DC, 20004, US

(21) Appl. No.: 12/083,360

(22) PCT Filed: Oct. 12, 2006
(86) PCT No.: PCT/US2006/040053

§ 371 Date: Apr. 17, 2009

Related U.S. Application Data
(60) Provisional application No. 60/725,286, filed on Oct. 12, 2005.

(51) Int. Cl. A61F 2/14; A61K 35/12

(52) U.S. Cl. 623/5.16; 424/428; 424/93.7

(57) ABSTRACT

The present invention provides a resorbable corneal button comprised of a biodegradable polymer which is capable of supporting the growth and expansion of endothelial cells on it surface for use in transplantation healthy corneal endothelial cells to cornea tissue in need of a transplant and a method of using same.

This is a national stage of PCT/US2006/040053 filed Oct. 12, 2006 and published in English, claiming benefit of U.S. provisional application No. 60/725,286, filed Oct. 12, 2005, and hereby incorporated by reference in its entirety.

### BACKGROUND OF THE INVENTION

1. Field of Invention

The present invention relates generally to a medical implant and method of implantation and, more particularly, for the treatment of cornea endothelial defects and disorders.

2. Description of Prior Art

### Corneal Endothelial Defects and Disorders

Endothelial dysfunction is the leading cause of corneal vision loss in the United States and is responsible for more than half of the 38,000 corneal transplants performed in this country each year [Aintablian, 2002 #1]. The cornea is the transparent, convex, outermost part of the eye and is the main refractive element of the visual system. Unlike most tissues in the body, the cornea contains no blood vessels to nourish or protect it against infection since the cornea must remain transparent to refract light properly, and the presence of even the tiniest blood vessels can interfere with this process. Instead, the cornea receives its nourishment from the tears and aqueous humor that fills the chamber behind it. The corneal tissue is arranged in five basic layers with the endothelium being the innermost layer. Endothelial cells are essential in keeping the cornea clear. Normally, fluid leaks slowly from inside the eye into the middle corneal layer (stroma). The endothelium's primary task is to pump this excess fluid out of the stroma. Without this pumping action, the stroma would swell with water, become hazy, and ultimately opaque. In a healthy eye, a perfect balance is maintained between the fluid moving into the cornea and fluid being pumped out of the cornea. Once endothelial cells are destroyed by disease, trauma or aging, they are lost forever. If too many endothelial cells are destroyed, edema and blindness ensue, with corneal transplantation as the only currently available therapy.

In the treatment of corneal endothelial disorders, it is common practice to replace the central portion of the cornea, including epithelium, stroma, Descemet's membrane and endothelium. To this end a full-thickness, cylindrical portion of the cornea is removed and replace by a similar part from a donor eye, a so called full-thickness keratoplasty. Although this procedure can provide excellent stromal graft clarity, it is plagued by the inherent problems of vertical stromal wounds that heal poorly and require surface corneal sutures. The latter cause irregular astigmatism and contribute to vision threatening situations such as ulceration, vascularization, and graft rejection.

It has been recognized that most of the corneal endothelial disorders could be treated by replacement of the Descemet's membrane together with the endothelium. To this end in 1993, WW Ko developed a technique of replacing the endothelium through a limbal incision. His results in an animal model led to further development by Gerrit Melles, who in 1998 published his results on posterior lamellar keratoplasty in the first human surgeries. The technique was developed in which the Descemet's membrane is removed through a sclerocorneal tunnel, together with said endothelium and a slice of the stroma on which the Descemet's membrane is carried. This is then replaced by a donor membrane with endothelium, on said slice of the stroma. The slice is cut from the stroma using a thin knife. The deep lamellar endothelial keratoplasty (DLEK) procedure avoids the inherent problems of PKP by allowing endothelial replacement without the need for surface corneal incisions or sutures and by maintaining the original, normal corneal topography.

There is a gross shortage of donor organs of all types on a global scale. Areas of applications include the replacement of nerve, visual, musculoskeletal, and soft tissues which may be severely damaged from combat-related injuries or disease. One notable example is blindness from injury or disease to the cornea. Cornea blindness ranks second to cataract as a cause of visual loss on the international scale. There are an estimated 10 million persons worldwide who suffer from cornea-associated visual impairment or corneal blindness. Once corneal endothelial cells are destroyed by disease, trauma or aging, they are lost forever. If too many endothelial cells are destroyed, edema and blindness ensue, with corneal transplantation as the only available therapy. However, a number of issues severely limit the success of this current treatment: lack of donor availability especially in countries where organ donation is culturally unacceptable, cost of tissue recovery, recent popularity of corrective laser surgery which precludes subsequent use of the cornea for transplantation, high rejection rate (20% of corneal allografts in adults and 50% of allografts in children end in allograft rejection), lack of a widely accepted corneal substitute, and that existing corneal prostheses do not integrate well into host tissue.

The use of polymers as a carrier for corneal endothelial cells has been investigated previously. See, for example, PCT/US04/032934, PCT/US04/032933, and PCT/US04/033194 to Lui and incorporated by reference herein as if set forth in their entireties.

The polymers can act as a permanent no-biodegradable carrier for the endothelial cell layers. The permanency of the polymer requires the removal of existing cell and connective tissue layers in the recipient. If the layers are not removed, there is a potential for a complication known as a dual anterior chamber.

Endothelial cells must form a tight single layer to function properly. Previous efforts have tried to encapsulate these cells into a biodegradable polymer, but the cells refuse to function unless cultured in a single layer.

### SUMMARY OF THE INVENTION

A primary challenge facing modern cornea transplant is the world-wide paucity of available donor cornea tissue for implantation. The device herein disclosed will remedy this shortage by utilizing a biodegradable polymer film and cultured cell combination as a replacement for donor corneas. The polymer film will act as a carrier for the cultured cell layer. Once implanted, the polymer film will dissolve, leaving the cell layer in its place.

By making the cell carrier biodegradable, only the cell layer is left behind. This method will be advantageous in situations where the connective tissue layer (Descemet's Membrane) is intact, but the cell layer has gaps.

The use of a biopolymer carrier to support the attachment, growth, and eventually as a vehicle to carrying the cells during transplantation is vital to the success of cell replacement therapy, particularly in the brain and the back of the eye, where cells derived from the neural crest origin is often damaged during the aging process. There are seven general classes of biopolymers: polynucleotides, polyamides, polysaccharides, polyisoprenes, lignin, polyphosphate and polyhydroxyalkanoates. See for example, U.S. Pat. No. 6,495,152. Biopolymers range from collagen IV to polyorganosiloxane compositions in which the surface is embedded with carbon particles, or is treated with a primary amine and optional peptide, or is co-cured with a primary amine- or carboxyl-containing silane or siloxane, (U.S. Pat. No. 4,822,741), or for example, other modified collagens are known (U.S. Pat. No. 6,676,969) that comprise natural cartilage material which has been subjected to defatting and other treatment, leaving the collagen II material together with glycosaminoglycans, or alternatively fibers of purified collagen II may be mixed with glycosaminoglycans and any other required additives. Such additional additives may, for example, include chondronectin or anchorin II to assist attachment of the chrondocytes to the collagen II fibers and growth factors such as cartilage inducing factor (CIF), insulin-like growth factor (IGF) and transforming growth factor (TGF).

It is therefore an object of the present invention to provide a resorbable corneal button support matrix having a polymer film coating, wherein the polymer film will be composed of hyaluronic acid. Hyaluronic acid is biodegradable, well tolerated by the eye, and can be formed into an optimal film for cell growth.

These and other objects of the invention, as well as many of the attendant advantages thereof, will become more readily apparent when reference is made to the following detailed description of the preferred embodiments.

### BRIEF DESCRIPTION OF THE DRAWINGS

FIG. 1 displays a front and side view of a preferred embodiment.

FIG. 2 displays an oblique view of an embodiment of the present invention.

FIG. 3 displays a schematic of cornea anatomy.

FIG. 4 shows a schematic of traditional DLEK procedure.

FIG. 5A shows a schematic of a modified DLEK procedure in which the endothelial cell layer is removed and the implant is placed directly on Descemet's Membrane.

FIG. 5B shows a schematic of a modified DLEK procedure in which the endothelial cell layer is not removed and the implant is placed on top of the remaining endothelial cells.

### DETAILED DESCRIPTION AND PREFERRED EMBODIMENTS

In describing a preferred embodiment of the invention specific terminology will be resorted to for the sake of clarity. However, the invention is not intended to be limited to the specific terms so selected, and it is to be understood that each specific term includes all technical equivalents which operate in a similar manner to accomplish a similar purpose.

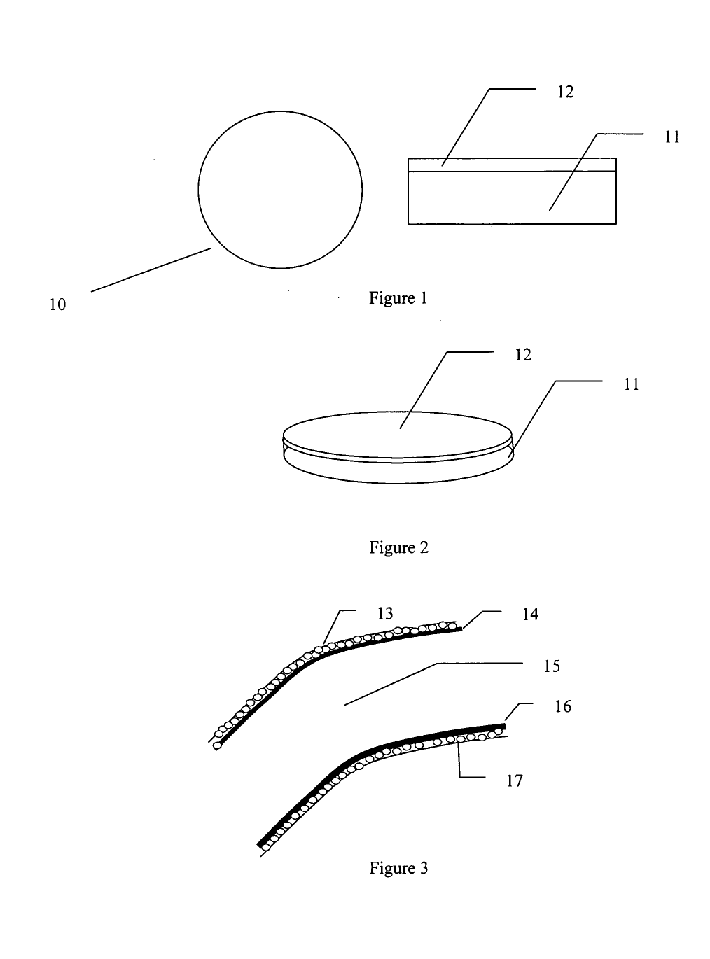

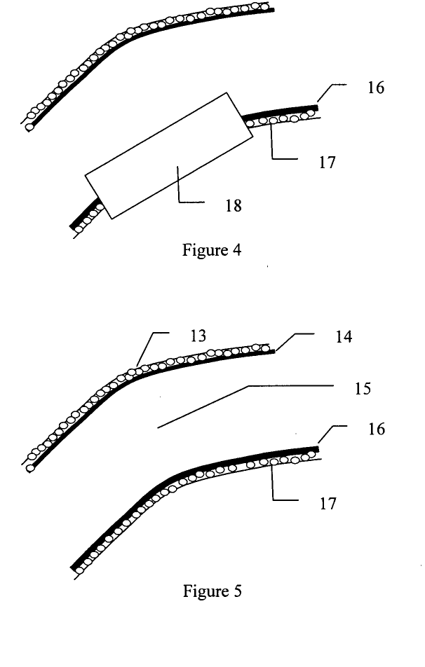

The preferred embodiment of the resorbable corneal button (RCB) device as shown in FIGS. 1-3, is designated generally as (10). In FIGS. 1 and 2, a preferred embodiment of the RCB device comprises a support matrix (11) which can be coated with a polymer film formed into a cylindrical shape with a layer of cultured cells (12) on top.

To improve the ability of the polymer in supporting cell growth or attachment, an attachment mixture comprising of one or more of the following will be embedded or incorporated into the support matrix (11) composition during synthesis: fibronectin at concentrations ranging from about to 500 g/ml of polymer gel, laminin at concentrations ranging from 1 to 500 g/ml of polymer gel, RGDS at concentrations ranging from 0.1 to 100 g/ml of polymer gel, bFGF conjugated with polycarbophil at concentrations ranging from 1 to 500 ng/ml of polymer gel, EGF conjugated with polycarbophil in concentrations ranging from 10 to 1000 ng/ml of polymer gel, NGF at concentrations of ranging from 1 to 1000 ng/ml of the polymer gel and heparin sulfate at concentrations ranging from 1 to 500 g/ml of polymer gel.

The approach of the present invention can also encompass the use of attachment proteins such as fibronectin, laminin, RGDS, collagen type IV, bFGF conjugated with polycarbophil, and EGF conjugated with polycarbophil. Polycarbophil is a lightly cross-linked polymer. The cross linking agent is divinyl glycol. Polycarbophil is also a weak poly-acid containing multiple carboxyl radicals which is the source of its negative charges. These acid radicals permit hydrogen bonding with the cell surface. Polycarbophil shares with mucin the ability to adsorb 40 to 60 times its weight in water and is used commonly as an over-the-counter laxative (Equalactin, Konsyl Fiber, Mitrolan, Polycarb) (Park H, et al., J. Control Release 1985; 2:47-57). Polycarbophil is a very large molecule and therefore is not absorbed. It is also non-immunogenic, even in the laboratory it has not been possible to grow antibodies to the polymer.

In one preferred embodiment of the present invention comprises a self-sustaining polymer which embeds or has incorporated within the polymer during it's synthesis, an attachment mixture comprising of one or more of the following: fibronectin, laminin, RGDS, bFGF conjugated with polycarbophil, EGF conjugated with polycarbophil, and heparin sulfate as described in PCT/US2004/032934. The polymer can be molded into any desired shape, such as the shape shown in FIG. 1, with the shape of a corneal button being preferred, and cultured human corneal endothelial cells will be seeded onto the concave surface and allowed to proliferate until confluent.

It is also contemplated that the present invention will utilize a self-sustaining biopolymer which can also be molded into half the thickness of the normal human cornea and covered with cultured human corneal endothelial cells for half-thickness transplantation using the DLEK procedure.

In a thin sheet or microparticle form, the coated biopolymer, in a preferred embodiment, is used as the support matrix for corneal endothelial cell growth and as a vehicle for cell delivery during a cell transplantation procedure.

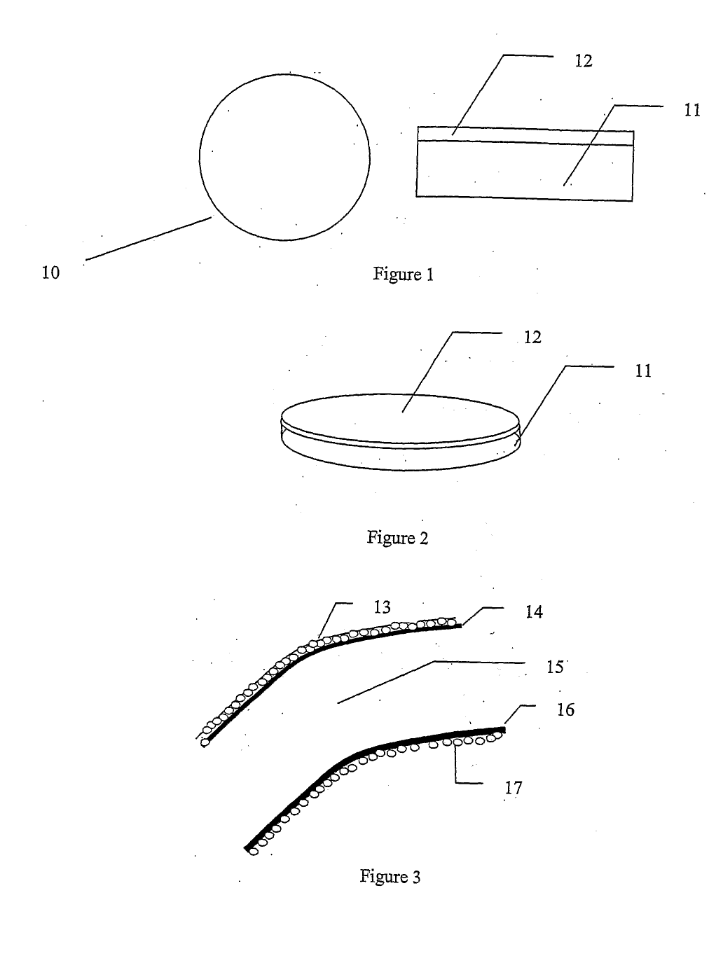

FIG. 3 shows an illustration of the cornea divided into sublayers. The first is a single layer of cells known as the Epithelium (13). Deep to the epithelium is Bowman's Layer (14) followed by the central Stroma (15). The posterior of the cornea is populated by Descemet's Membrane (16) and the last layer of cells known as the endothelium (17).

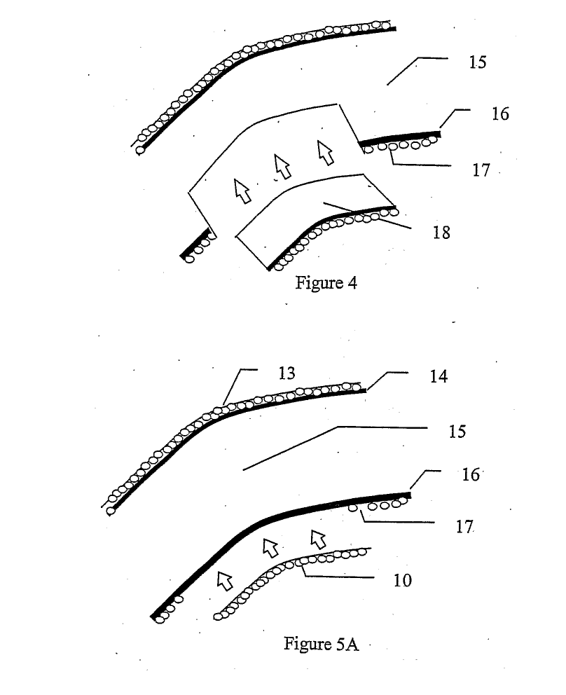

FIG. 4 depicts a traditional DLEK procedure. In the procedure, Descemet's Membrane (16), part of the Stroma (15), and the Endothelium (17) are removed and replaced by the implant (18). The implant (18) contains endothelial cells, Descemet's Membrane, and part of the Stroma (15). In a DLEK procedure, the surgeon uses special instruments to enter the white of the eye (sclera) and “tunnel” into the diseased cornea. The back portion of the cornea is then removed and replaced by a similar piece of healthy graft tissue from a corneal donor. Although only a small piece of cornea is actually replaced, the graft will help keep the entire cornea clear.

DLEK has several advantages over conventional transplant surgery. No stitches are placed in the cornea. In clinical studies, this has resulted in significantly less astigmatism after surgery and faster recovery of vision. In general, fewer follow-up exams are necessary because there are no corneal stitches to be removed. Ongoing studies are also examining whether corneal transplant rejection is less likely with DLEK than conventional transplants.

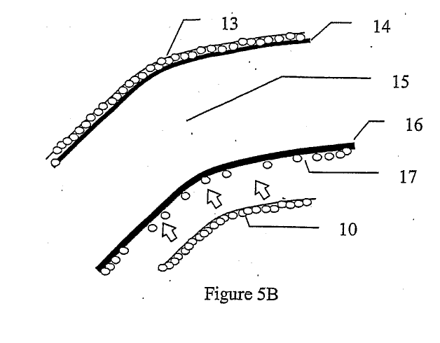

FIGS. 5A and 5B present the modified procedure of the present invention, Descemet's Membrane (16) is not removed, and the endothelial cell layer (17) may or may not be removed. In the case of Fuch's dystrophy, the existing endothelial cell layer may be damaged beyond repair. In this case, it must be removed entirely. In other situations, the endothelial cell layer may be depleted, but only slightly damaged. In these situations, the remaining endothelial cells are not removed and the implant is placed on top of them. FIG. 5A illustrates the situation in which the endothelial cell layer (17) is removed and the implant (10) is placed directly on Descemet's Membrane (16). FIG. 5B illustrates the situation in which the endothelial cell layer (17) is not removed and the implant (10) is placed on top of the remaining endothelial cells.

### Use of the Device

In a preferred embodiment, a thin polymer layer will be used for the support matrix and formed from hyaluronic acid. Endothelial cells will be harvested from the patient needing transplant. These cells will be grown, expanded, and seated on the polymer layer using the techniques described in patent application PCT/US04/32933 to form the RCB. Once the cells reach confluence on the polymer, the RCB is ready to be implanted.

A standard DLEK, corneal-scleral incision is made to access the anterior chamber. In the preferred embodiment, the existing endothelial cells are not removed and the RCB is placed on top. Once placed into the anterior chamber, the cells of the RCB will begin to pump. The suction action created by the cells will hold the RCB in intimate proximity to the existing cornea.

Once the RCB is seated on the cornea, hyaluronase is injected into the anterior chamber and the incision is closed. The hyaluronase acts as an enzyme catalyst to speed the decomposition of the hyaluronic acid polymer disk support matrix. In the preferred embodiment, the disk dissolves within 24 hours, leaving the new endothelial cells firmly attached to the patient's cornea.

It is to be understood that the dimensions for the size and shape of cuts made in the recipient and donor corneal tissues are merely representative of the type of surgery which can be done. Thus, variations in the dimensions and shape of the pocket, flap, cap, and corneal donor or recipient disks are expected, all keeping within the scope of the present invention.

It is contemplated generally, that any type of resorbable polymer known in the art, can be used as the support matrix for the RCB. The polymer can be placed directly on top of existing endothelial layer or existing layer can be scraped off first. In an alternated embodiment, the existing endothelial layer can be stunned (chemically or using RF current) for 24 hrs to allow resorption of polymer film and remove risk of dual anterior chamber.

In an alternate embodiment, the polymer carrier could be comprised of mammalian amniotic membranes or a combination of amniotic membrane and collagen. See for example, U.S. patent application 2005/0214259 to Sano et al., which teaches that corneal endothelial cells can be collected, and then cultured and proliferated in vitro. A cell suspension with high cell density can be produced by subculturing the proliferated cells and subjecting them to appropriate centrifugation. Then, as a substrate (carrier), amniotic membrane containing collagen as a main component was employed, and the cell suspension was planted thereon and cultured for a predetermined time. As a result, a single layered cell layer, in which cells derived from the corneal endothelial cells, can have a similar morphology to that of the living body, can be formed. It has been found that these cell layers can have the equivalent cell density to the corneal endothelial cells of a living body and had a configuration in which hexagonal shaped cells were regularly layered to form a single layer structure.

A variety of biomaterials have been used to treat and repair corneal and ocular defects and injuries, and it is contemplated that many are suitable for use as a support matrix for the RCB. For example, the corneal extracellular matrix is rich in collagen and glycosaminoglycans (Robert et al 2001, Pathol Biol (Paris); 49(4):353-63). The glycosaminoglycan, hyaluronan, has been found to improve corneal epithelial wound healing in rat and rabbit models, as assessed by evaluation of the stromal and endothelial layers (Nakamura et al 1997, Exp Eye Res; 64(6):1043-50; Chung et al 1999, Ophthalmic Res; 31(6):432-9). Tseng and others have pioneered the use of amniotic membrane in the treatment of a variety of ocular disorders (U.S. Pat. No. 6,152,142). The amniotic membrane is polarized, with a ‘stromal’ side and a ‘basement membrane’ side. The stromal side contains collagens I and III and fibronectin with a basal lamina distribution of collagen type IV, laminin and heparin sulfate proteoglycan. The basement membrane side of the amniotic membrane supports epithelial cell growth, while the stromal side supports the growth of fibroblasts in a manner similar to collagen. The amniotic membrane is isolated from the human placenta, cryopreserved, and then used for the surgical repair of intra-ocular disorders.

The mechanism of action of the amniotic membrane remains incompletely understood. However, there is in vitro evidence that the presence of amniotic membrane in culture suppresses the expression of TGF by fibroblasts (Lee et al 2000, Curr Eye Res; 20(4):325-334) and interleukin 1.alpha. and interleukin 1 by epithelial cells (Solomon et al 2001, Br J Opthalmol; 85(4): 444-449).

The amniotic membrane has also been used successfully to treat a wide range of corneal and ocular defects. For example, deep corneal and scleral ulcers have been treated by the use of multi-layers of the amniotic membrane to fill stromal layer, basement membranes, and as a wound cover (Hanada et al 2001, Am J Opthalmol; 131(3):324-31). Amniotic membrane was found to reduce stromal inflammation and ulceration in HIV-1 keratitis, an immune mediated disease (Heiligenhaus et al 2001, Invest Opthalmol Vis Sci; 42(9):1969-1974). Severe neurotrophic corneal ulcers also have been treated with amniotic membranes (Chen et al 2000, Br J Opthalmol; 84(8): 826-833). Amniotic membrane restored the corneal and conjunctival surfaces and reduced limbal stromal inflammation resulting from acute chemical or thermal burns (Meller et al 2000, Opthalmology; 107(5): 980-989). Amniotic membrane was used as an alternative to limbal autograft or allograft in patients with partial limbal stem cell deficiency (Anderson et al 2001, Br J Opthalmol; 85(5):567-575). Amniotic membranes have also been used in surgical treatment of pterygia, a wing-like fold of membrane extending from the conjunctiva to the cornea, with attachments to the sclera (Solomon et al 2001, Opthalmology: 108(3):449-460). Amniotic membranes were used to treat late onset glaucoma filtering bed leaks as an alternative to conjunctiva with success (Budenz et al 2000, Am J Opthalmol; 130(5): 580-588; Barton et al 2001, Invest Opthalmol Vis Sci; 42(8):1762-1768) as well as to improve recovery of a stable corneal epithelium and reduce ocular pain when used in the surgical treatment of band keratopathy, the deposition of calcium in the corneal basement membrane secondary to sarcoidosis, chronic uveitis and other causes (Anderson et al 2001, Cornea; 20(4): 354-361).

It is also contemplated that other substrates may be used with the corneal endothelial cells as a support matrix for the RCB. In another embodiment chitosan can be used as a support matrix.

Chitosan is a cationic biopolymer comprising glucosamine and N-acetyl glucosamine that has bioadhesive properties and has been shown to improve the systemic bioavailability of certain drug compounds across mucosal surfaces, such as the nasal cavity (see Illum, Drug Discovery Today, 7:1184-1189 (2002)).

By the term “chitosan” we include all derivatives of chitin, or poly-N-acetyl-D-glucosamine, including all polyglucosamines and oligomers of glucosamine materials of different molecular weights, in which the greater proportion of the N-acetyl groups have been removed through hydrolysis (deacetylation). In accordance with the present invention, the degree of deacetylation, which represents the proportion of N-acetyl groups which have been removed through deacetylation, should preferably be in the range of about 40-97%, more preferably in the range of about 60-96% and most preferably be in the range of about 70-95%.

The chitosan, chitosan derivative or salt used in the present invention should preferably have a molecular weight in the range of about 10,000 to 1,000,000 Da, more preferably in the range of about 15,000 to 750,000 Da and most preferably in the range of about 20,000 to 500,000 Da.

Salts of chitosan are suitable for use in the present invention. Salts with various organic and inorganic acids are suitable. Such suitable salts include, but are not limited to, the nitrate, phosphate, glutamate, lactate, citrate, hydrochloride and acetate salts. Preferred salts are the hydrochloric acid and glutamic acid salts.

Chitosan derivatives and their salts are also suitable for use in this invention. Suitable chitosan derivatives include, but are not limited to, esters, ethers or other derivatives formed by bonding acyl and/or alkyl groups with the hydroxyl groups, but not the amino groups of chitosan. Examples include O-alkyl ethers of chitosan and O-acyl esters of chitosan. Modified chitosans, such as those conjugated to polyethylene glycol may be used in the present invention. Conjugates of chitosan and polyethylene glycol are described in International patent application publication WO99/01498.

Chitosans suitable for use in the present invention may be obtained form various sources, including Primex, Haugesund, Norway; NovaMatrix, Drammen, Norway; Seigagaku America Inc., MD, USA; Meron (India) Pvt, Ltd., India; Vanson Ltd, VA, USA; and AMS Biotechnology Ltd., UK. Suitable derivatives include those that are disclosed in Roberts, Chitin Chemistry, MacMillan Press Ltd., London (1992).

The support matrix or “carrier” for the RCB can also be comprised of a water-containing polymer gel containing chitosan, and the surface of the water-containing gel is coated with collagen and/or alginic acid. Further, the carrier for RCB of the present invention according to another aspect could comprise a gel layer containing chitosan and an inorganic layer adjacently provided to the gel layer.

The term “carrier for the RCB” used in the specification means an element that can serve as a carrier or support during cell culture, and this term should not be construed any limiting way. For example, a carrier for cell culture is described in Japanese Patent Unexamined Publication (KOKAI) No. 2001-120267, in which an alginic acid gel layer and an extracellular matrix component gel layer as a cell adhesion component gel layer are laminated on a porous membrane, and the carrier for the RCB of the present invention can be used for culture in the same technical field similar as that of the carrier for cell culture described in the above patent document.

The term “gel containing chitosan” means a gel that contains chitosan gel as a main component. The water-containing polymer gel containing chitosan means a water-containing polymer gel containing “chitosan gel” as a main component (in the specification, a water-containing polymer gel containing chitosan may also be henceforth referred to as a “chitosan gel”). As the chitosan gel, a gel can be used which does not dissolve in a neutral region in which cell culture is performed. For example, a chitosan gel formed as a gel, which does not dissolve in a neutral region in which cell culture is performed, by neutralizing the amino groups in the molecules of chitosan, a chitosan gel formed as a gel by salt formation of chitosan and an organic polymer compound having an anionic residue, a chitosan gel formed as a gel by crosslinking with a crosslinking agent and the like can be utilized. As the organic polymer compound having an anionic residue, for example, natural or synthetic polymer compounds such as polyaspartic acid, alginic acid, dextran sulfate, chondroitin sulfate, and polystyrenesulfonic acid can be used. Examples of the crosslinking agent include compounds having two or more groups that react with amino group or hydroxyl group such as glutaraldehyde, divinyl sulfone, and halogenated triazine, compounds having two or more carboxylic acid groups which are made into active esters beforehand and the like.

Chitosan (poly D-glucosamine) can be obtained by heating chitin (poly-N-acetyl-D-glucosamine) with a concentrated alkali solution or subjecting chitin to potassium fusion, and then deacetylating the resultant. Any chitosan can be used for manufacture of the carrier for the RCB of the present invention. For example, from a viewpoint of formation of a membrane having a high membrane strength, preferred is chitosan having a deacetylation degree of from 60 to 100%, and providing a solution viscosity of from 10 to 10000 cP when dissolved at 0.5 mass % in 1 mass % aqueous acetic acid solution. More preferred is chitosan having a deacetylation degree of from 70 to 100%, and providing the solution viscosity of from 40 to 5000 cP.

A method of successively coating various other polymer compounds including collagen, alginic acid, and chitosan on the gel surface for use in the carrier of the RCB is not particularly limited. The layer-by-layer method (Gero Decher, Science, No. 277, pp. 1232-1237, Aug. 29, 1997,) is preferably used, for example. The layer-by-layer method comprises repeating immersion of a membrane in an aqueous solution of any one of various polymer compounds, subsequent washing with water and immersion in another polymer compound. For producing the carrier for the RCB of the present invention, surface modification for the surface of the water-containing polymer gel containing chitosan can be performed for both sides or one side of the water-containing polymer gel. For performing the modification for one side, a method of attaching a cover on one side, during the aforementioned modification method based on application or the aforementioned modification method based on immersion, is preferably used so that said side is not brought into contact with an immersion solution. For the gelation, a gelling agent may be used, if needed.

In another embodiment, the support matrix for the RCB could be made from a film derived from collagen matrix that is cross-linked. Such material can be made using a process comprising the steps of: procuring a collagen-based biological tissue from a mammal; treating the biological tissue with polyepoxy compound to obtain a biological tissue with cross-linked collagen structure; decellularizing the biological tissue thus obtained to give a cell-free tissue; and, immersing the cell-free tissue in a cryoprotective solution containing hyaluronic acid and freeze-drying the said tissue. The collagen-based tissue includes, but not limited these to, preferably fascia, amnion, placenta or skin of mammals. Polyepoxy compound includes, but not limited these to, preferably polyglycerol polyglycidyl ether, polyethylene glycol glycidyl ether, or other commercially available polyepoxy compounds. Preferably, 1-7% (w/v) of polyepoxy compound is treated on biological tissue at the condition of pH 8-11, at 30-45 C for 10-20 hours. Further, the freeze-dried cell-free tissue is preferably pulverized by physical means, for example, cryo-pulverization is carried out in a pulverizer under an environment of liquid nitrogen, to protect it from the damage by heat generated in the course of processing. The method may further comprise a step of pulverizing the freeze-dried cell-free tissue into smaller ones under an environment of liquid nitrogen before the cryo-pulverization or the steps of hydrating the freeze-dried cell-free tissue and cutting the hydrated tissue.

A variety of cross-linking techniques are known to stabilize the structure of collagen, while maintaining the mechanical strength and unique properties of collagen tissues for transplantation. In addition to the cross-linking techniques, studies on decellularizing technique has been actively performed to reduce the immune-rejection against transplanted graft during transplantation, to proliferate cells in the graft and to develop new biomaterials for tissue engineering. Many researches related to glutaraldehyde have been conducted to increase the stability of tissue structure, which revealed a serious problem of the high toxicity of glutaraldehyde in human bodies. In this regard, alternative techniques for the cross-linking of collagen tissue have been explored in the art, one of which is cross-linking technique of collagen tissue using polyepoxy compounds.

Cross-linking has been known in the art for years and there are various methods both chemical and physical (irradiation) methods. Exemplary chemical cross-linking agents of choice known in the art have been glutaraldehyde and other related non-physiological agents. These cross-linking agents react with amino acid residues of the collagen molecule to form intermolecular cross-links. However, these harsh agents may have negative effects on the biocompatibility and biological activity of cross-linked collagen-based bioproducts that are caused by alterations in the conformation of the collagen molecule and leaching out of the cross-linking agents. Thus, collagen products cross-linked by non-physiological agents are poorly accepted by and integrated within the host tissues. Furthermore, localized inflammation and more complex systemic reactions are disadvantageous side effects of glutaraldehyde cross-linked collagen products.

U.S. Pat. No. 4,971,954 to Brodsky et al. discloses the use of D(−)Ribose or other reducing physiological sugars as physiological agents for cross-linking collagen matrices by the process of glycation. However, the method disclosed by Brodsky et al. is efficient when the collagenous substrate consists of native collagen fibers, but is only partially effective for collagen matrices produced from reconstituted fibrillar collagen, particularly when the collagen is atelopeptide collagen. Atelopeptide collagen is produced by pepsin-solubilization of native collagen. Since pepsin cuts off the telopeptides of the collagen molecule which are antigenic, pepsin-solubilized collagen is the most utilized form of collagen in the biomedical industry.

A further contemplated support matrix for the RCB is that derived from adipocytes or fat cells. Adipose-derived stem cells or “adipose-derived stromal cells” refer to cells that originate from adipose tissue. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably the adipose tissue is human. A convenient source of adipose tissue is from liposuction surgery, however, the source of adipose tissue or the method of isolation of adipose tissue is not critical to the invention.

Adult human extramedullary adipose tissue-derived stromal cells represent a stromal stem cell source that can be harvested routinely with minimal risk or discomfort to the patient. Pathologic evidence suggests that adipose-derived stromal cells are capable of differentiation along multiple lineage pathways. Adipose tissue is readily accessible and abundant in many individuals. Obesity is a condition of epidemic proportions in the United States, where over 50% of adults exceed the recommended BMI based on their height.

It is well documented that adipocytes are a replenishable cell population. Even after surgical removal by liposuction or other procedures, it is common to see a recurrence of adipocytes in an individual over time. This suggests that adipose tissue contains stromal stem cells that are capable of self-renewal

Adipose tissue offers many practical advantages for tissue engineering applications such as the RCB of the present invention. First, it is abundant. Second, it is accessible to harvest methods with minimal risk to the patient. Third, it is replenishable. While stromal cells represent less than 0.01% of the bone marrow's nucleated cell population, there are up to 8.6×10₄ stromal cells per gram of adipose tissue (Sen et al 2001, Journal of Cellular Biochemistry 81:312-319). Ex vivo expansion over 2 to 4 weeks yields up to 500 million stromal cells from 0.5 kilograms of adipose tissue. These cells can be used immediately or cryopreserved for future autologous or allogeneic applications.

Methods for the isolation, expansion, and differentiation of human adipose tissue-derived cells have been reported. See for example, Burris et al 1999, Mol Endocrinol 13:410-7; Erickson et al 2002, Biochem Biophys Res Commun. Jan. 18, 2002; 290(2):763-9; Gronthos et al 2001, Journal of Cellular Physiology, 189:54-63; Halvorsen et al 2001, Metabolism 50:407-413; Halvorsen et al 2001, Tissue Eng. 7(6):729-41; Harp et al 2001, Biochem Biophys Res Commun 281:907-912; Saladin et al 1999, Cell Growth & Diff 10:43-48; Sen et al 2001, Journal of Cellular Biochemistry 81:312-319; Zhou et al 1999, Biotechnol. Techniques 13: 513-517. Adipose tissue-derived stromal cells are obtained from minced human adipose tissue by collagenase digestion and differential centrifugation [Halvorsen et al 2001, Metabolism 50:407-413; Hauner et al 1989, J Clin Invest 84:1663-1670; Rodbell et al 1966, J Biol Chem 241:130-139]. Others have demonstrated that human adipose tissue-derived stromal cells can differentiate along the adipocyte, chondrocyte, and osteoblast lineage pathways [Erickson et al 2002, Biochem Biophys Res Commun. Jan. 18, 2002; 290(2): 763-9; Gronthos et al 2001, Journal of Cellular Physiology, 189:54-63; Halvorsen et al 2001, Metabolism 50:407-413; Halvorsen et al, 2001, Tissue Eng. Dec. 7, 2001(6):729-41; Harp et al 2001, Biochem Biophys Res Commun 281:907-912; Saladin et al 1999, Cell Growth & Diff 10:43-48; Sen et al 2001, Journal of Cellular Biochemistry 81:312-319; Zhou et al 1999, Biotechnol. Techniques 13: 513-517; Zuk et al 2001, Tissue Eng. 7: 211-228.

WO 00/53795 to the University of Pittsburgh and The Regents of the University of California and U.S. patent application Ser. No. 2002/0076400 assigned to the University of Pittsburgh, disclose adipose-derived stem cells and lattices substantially free of adipocytes and red blood cells and clonal populations of connective tissue stem cells. The cells can be employed, alone or within biologically-compatible compositions, to generate differentiated tissues and structures, both in vivo and in vitro. Additionally, the cells can be expanded and cultured to produce hormones and to provide conditioned culture media for supporting the growth and expansion of other cell populations. In another aspect, these publications disclose a lipo-derived lattice substantially devoid of cells, which includes extracellular matrix material form adipose tissue. The lattice can be used as a substrate to facilitate the growth and differentiation of cells, whether in vivo or in vitro, into anlagen or mature tissue or structures. Neither publication discloses adipose tissue derived stromal cells that have been induced to express at least one phenotypic or genotypic characteristic of an intra-ocular stromal cell.

U.S. Pat. No. 6,429,013 assigned to Artecel Sciences discloses compositions directed to an isolated adipose tissue-derived stromal cell that has been induced to express at least one characteristic of a chondrocyte. Methods are also disclosed for differentiating these cells.

As a non-limiting example, in one method of isolating adipose tissue derived stromal cells, the adipose tissue is treated with collagenase at concentrations between 0.01 to 0.5%, preferably 0.04 to 0.2%, most preferably 0.1%, trypsin at concentrations between 0.01 to 0.5%, preferably 0.04 to 0.04%, most preferably 0.2%, at temperatures between 25 C to 50 C., preferably between 33 C to 40 C., most preferably at 37 C, for periods of between 10 minutes to 3 hours, preferably between 30 minutes to 1 hour, most preferably 45 minutes. The cells are passed through a nylon or cheesecloth mesh filter of between 20 μm to 800 μm, more preferably between 40 to 400 μm, most preferably 70 μm. The cells are then subjected to differential centrifugation directly in media or over a Ficoll or Percoll or other particulate gradient. Cells can be centrifuged at speeds of between 100 to 3000×g, more preferably 200 to 1500×g, most preferably at 500×g for periods of between 1 minute to 1 hour, more preferably 2 to 15 minutes, most preferably 5 minutes, at temperatures of between 4 C to 50 C, preferably between 20 C to 40 C, most preferably at 25 C.

It is known in the art that alginate gels may be formed by mixing with dicovalent cations, like Ca2+ or Mg2+ to form an ionic gel. This gel can lose mechanical strength and dissolve quickly due to the loss of ions to surrounding medium. See, Jon A. Rowley, Gerard Madlambayan, David J. Mooney, Biomaterials 20 (1999) 45-53. This type of gel can also be used for the carrier of the RCB.

It is also contemplated that gelatin and its derivatives can be used as a resorbable support matrix for RCB. The use of gelatin in similar settings can be found in Krishna Burugapalli, Veena Koul, Amit K. Dinda, J Biomed Mater Res 68A: 210-218, 2004; and Hye-Won Kang, Yasuhiko Tabata, Yoshito Ikada, Biomaterials 20 (1999) 1339-1344.

It is also contemplated that a composition comprising carboxymethylcellulose and its derivatives can be used and a resorbable support matrix for the RCB. The use of crosslinked carboxymethylcellulose in tablet manufacture is well known from published literature such as Wan and Prasad, Effect of Microcrystalline Cellulose and Crosslinked Sodium Carboxymethylcellulose on the Properties of Tablets with Methyl cellulose as a Binder, International Journal of Pharmaceutics, 41, (1988) 159-167. Indeed it is known in the art to use an acid crosslinked carboxymethylcellulose identified as croscarmellose sodium, type A, NF or crosslinked polyvinyl pyrrolidone or sodium starch glyconate in the manufacture of oral or gastric disintegrating tablets. Such compositions can be readily adapted for use in the present invention by one of ordinary skill without undue experimentation.

Prior to implantation of the RCB, the endothelial cell layer can be stunned. If the existing endothelial cells are not stunned or removed, they have the potential to cause complications. The cells will continue to pump fluid from the stroma. This fluid pumping action might cause fluid to collect between the cornea and the RCB making it difficult to seat firmly against the cornea. Stunning the cells will allow the new layer of cells time to come to confluence on the cornea before the pumping from the existing cells cause problems.

It is contemplated that methods to stun the endothelial cells of the present invention include exposure to various wavelengths of radio frequency radiation (RF), UV, irradiation with nuclear radiation such as gamma radiation, as well as chemical means such as trypsinization, acids, bases, hypoosmotic solutions, buffers with low ion (Mg, Na, Ca, K,) etc.

Prior to implantation of the RCB, the endothelial cell layer can be removed using vacuum action or physical scraping.

Having described the invention, many modifications thereto will become apparent to those skilled in the art to which it pertains without deviation from the spirit of the invention as defined by the scope of the appended claims. All references recited herein are incorporated by reference in their entireties as if fully set forth in the specification.

### REFERENCES

1. Ko W W, Feldman S T, Frueh B E, et al. Experimental posterior lamellar transplantation of the rabbit cornea. Invest Opthalmol Vis Sci. 1993; 34(suppl): 1102.
2. Melles G R, Eggink F A, Lander F, et al. A surgical technique for posterior lamellar keratoplasty. Cornea. 1998; 17:618-626.
3. Melles G R, Lander F, van Dooren B R, et al. Preliminary clinical results of posterior lamellar keratoplasty through a sclerocorneal pocket incision. Opthalmology. 2000; 107:1850-1856.
4. Terry M A, Ousley P J. Endothelial replacement without surface corneal incisions or sutures: topography of the deep lamellar endothelial keratoplasty procedure. Cornea. 2001; 20:14-18.
5. Terry M A, Ousley P J. Deep lamellar endothelial keratoplasty in the first United States patients: early clinical results. Cornea. 2001; 20:239-243.
6. Terry M A, Ousley P J. Replacing the endothelium without surface corneal incisions or sutures: first US clinical series with the deep lamellar endothelial keratoplasty procedure. Opthalmology. 2003; 110:755-764.
7. Terry M A. Endothelial replacement: the limbal pocket approach. Opthalmol Clin North Am. 2003; 16:103-112.
8. Terry M A, Ousley P J. Corneal endothelial transplantation: advances in the surgical management of endothelial dysfunction. Contemporary Opthalmology. 2002; 1:26:1-9.
9. Terry M A, Ousley P J. Rapid visual rehabilitation with deep lamellar endothelial keratoplasty. Cornea. 2004; 23:143-153.

1. A resorbable corneal button useful in repairing a cornea comprising:
a) a biodegradable polymer support matrix having a top and bottom side, capable of supporting growth of endothelial cells;
b) a layer of viable endothelial cells suitable for transplantation disposed on the top side; and
c) said resorbable corneal button being well tolerated by the eye when implanted into the eye.

2. The resorbable corneal button of claim 1, wherein the biodegradable polymer support matrix is selected from the group consisting of hyaluronic acid, amniotic membrane, chitosan, cross-linked collagen, alginates, adipose tissue, gelatins, carboxymethylcellulose and combinations thereof.

3. The resorbable corneal button of claim 1, wherein the endothelial cells are corneal endothelial cells.

4. The resorbable corneal button of claim 2, wherein said biodegradable polymer support matrix comprises hyaluronic acid and wherein the endothelial cells are corneal endothelial cells.

5. The resorbable corneal button of claim 2, wherein said biodegradable polymer support matrix comprises cross-linked collagen and wherein the endothelial cells are corneal endothelial cells.

6. A method for repairing a cornea comprising:
a) obtaining a polymer support matrix;
b) harvesting corneal endothelial cells from a patient needing a transplant or using banked endothelial cells;
c) making a resorbable corneal button by growing and expanding the harvested or banked corneal endothelial cells on the surface of said polymer support matrix until the cells reach confluence;
d) performing a standard DLEK, corneal-scleral incision on the eye in need of a transplant in order to access the anterior chamber of the cornea;
e) implanting the resorbable corneal button on top of the existing endothelial cells in the anterior chamber of the cornea so that the transplanted endothelial cells are in intimate contact with the cells of the anterior chamber of the cornea;
f) injection of hyaluronase into the anterior chamber; and
g) closing the incision of the eye.

7. The method for repairing a cornea of claim 6, wherein the endothelial cells that are in the location of the cornea which will receive the resorbable corneal button are stunned prior to the implantation step e).
